(19) United States
(12) Patent Application Publication
(10) Pub. No.: US 2025/0010066 A1
(43) Pub. Date: Jan. 9, 2025

(54) ADJUSTABLE NEEDLE ELECTRODES FOR MEDICAL PROCEDURES AND ASSOCIATED SYSTEMS AND METHODS

(71) Applicant: BOSTON SCIENTIFIC SCIMED, INC., MAPLE GROVE, MN (US)

(72) Inventors: Serena Scott, Worcester, MA (US); Timothy A. Ostroot, Cokato, MN (US); Laura Emily Sabban, Plainville, MA (US)

(73) Assignee: BOSTON SCIENTIFIC SCIMED, INC., MAPLE GROVE, MN (US)

(21) Appl. No.: 18/762,905

(22) Filed: Jul. 3, 2024

Related U.S. Application Data
(60) Provisional application No. 63/524,999, filed on Jul. 5, 2023.

(51) Int. Cl. A61N 1/32; A61B 18/00; A61B 18/14

(52) U.S. Cl. CPC A61N 1/327; A61B 18/1477; A61B 2018/00083; A61B 2018/00577; A61B 2018/00589; A61B 2018/00613; A61B 2018/00916; A61B 2018/1425; A61B 2018/1475; A61B 2218/007

(57) ABSTRACT

Bipolar or monopolar adjustable energy-delivering assemblies. The assemblies are configured for transluminal (e.g., endoscopic) delivery within a patient. A first energy-delivering member defines a first electrode portion formed of an electrically-conductive material so that energy may be delivered to the first energy-delivering member to create an energy field along the first electrode portion to apply to a target site within a patient. The first energy-delivering member may define a lumen therethrough allowing delivery of materials distally therethrough to a target site and/or allows materials from the target site to be aspirated proximally therethrough. Optionally, a second energy-delivering member and a second insulation member form a second electrode portion. Optionally, either or both electrode portions may be adjustable. Additionally or alternatively, the first electrode portion and the second electrode portion are adjustable with respect to each other.

### CROSS-REFERENCE TO RELATED APPLICATIONS

This application claims the benefit of priority under 35 U.S.C. § 119 to U.S. Provisional Application No. 63/524,999, filed Jul. 5, 2023, the entire disclosure of which is hereby incorporated by reference herein for all purposes.

### FIELD

The present disclosure relates generally to the field of medical devices, assemblies, systems, and methods used in applying energy to a patient, such as for therapeutic purposes. More particularly, the present disclosure relates to the field of medical devices, assemblies, systems, and methods for applying electrical energy, such as therapeutic electrical pulses, to a patient. Even more particularly, the present disclosure relates to various devices, assemblies, systems, and methods for electroporation treatment. And, even more particularly, the present disclosure relates to adjustable length medical treatment devices, assemblies, and systems applying energy, such as electrical energy such as for electroporation, and associated methods.

### BACKGROUND

Various devices, assemblies, and systems exist for energy-based medical treatment or therapeutic protocols. Currently, bipolar devices (with two electrodes) may be inserted percutaneously or endoscopically into a patient. Once positioned within the target site (e.g., a tumor), the device is activated, such as by generating an electric field between and/or around the two electrodes of the device, for various therapeutic procedures. For instance, energy may be applied to perform radiofrequency ablation (RFA), electroporation, and/or irreversible electroporation (IRE) as a mode of treating various conditions and/or diseases by interrupting and/or changing the nature of biological cellular matter. Irreversible electroporation energy applied by the device causes pores to open within cell membranes near the device. The electric field disrupts homeostasis, killing the cells. Radiofrequency ablation causes thermal coagulation of the tissue.

Bipolar electroporation devices generally produce less muscle contractions than monopolar devices which have a single active electrode and a return grounding pad positioned on the patient's skin. However, in contrast with monopolar devices, bipolar devices may have lower ablation volumes, arcing between electrodes, greater complexity, and multiple layers of electrodes and insulation between them to fit into a small diameter needle. In contrast with bipolar devices, monopolar devices may be considered simpler in nature, may create a larger ablation volume, may have reduced risks of arcing, and may afford space for other features or structures or devices, such as increased insulation thickness. Thicker insulation afforded by monopolar devices, in turn, may allow for improved dielectric strength, improved electrical safety, and the ability to be used at higher voltages for higher ablation volumes.

An adjustable length electrode (either monopolar or bipolar) may provide various benefits, such as ensuring that ablation volumes are well-conformed to target sites (e.g., tumor volumes), ensuring full ablation of the tumor volume, and helping to avoid ablation of non-targeted nearby anatomy, such as healthy tissue (e.g., pancreatic tissue, GI tract walls, etc.) and/or blood vessels. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an energy-delivering assembly includes a first energy-delivering member having a sharp distal tip configured to puncture tissue, and a lumen defined therethrough and having a distal opening adjacent the sharp distal tip for delivery of materials distally through the lumen and out the distal opening thereof and/or for aspiration of materials into the distal opening of the lumen and proximally through the lumen; and a first insulation member extending over the first energy-delivering member and having a distal end. In some aspects, the first electrode portion of the energy-delivering assembly is defined along the first energy-delivering member between the sharp distal tip thereof and the distal end of the first insulation member.

In some aspects, the first electrode portion is adjustable by moving the first insulation member with respect to the first energy-delivering member.

In some aspects, the first electrode portion and the first insulation member have substantially the same outer diameters.

In some aspects, the energy-delivering assembly further includes a second energy-delivering member positioned over the first insulation member and having a distal end; and a second insulation member extending over the second energy-delivering member and having a distal end. In some aspects, a second electrode portion of the energy-delivering assembly is defined along the second energy-delivering member between the distal end thereof and the distal end of the second insulation member. In some aspects, at least one of the first energy-delivering member, the first insulation member, the second energy-delivering member, or the second insulation member is movable with respect to another of the first energy-delivering member, the first insulation member, the second energy-delivering member, or the second insulation member to adjust at least one of the first electrode portion or the second electrode portion. In some aspects, the first insulation member is movable with respect to the first energy-delivering member to adjust the first electrode portion. In some aspects, the second energy-delivering member is movable with respect to the first insulation member. In some aspects, the second insulation member is movable with respect to the second energy-delivering member. In some aspects, the second energy-delivering member is movable with respect to the first insulation member. In some aspects, the second insulation member is movable with respect to the second energy-delivering member. In some aspects, the first energy-delivering member and the first insulation member are fixed with respect to each other, and the second energy-delivering member and the second insulation member are fixed with respect to each other. In some aspects, the second energy-delivering member is distally advanceable to move the distal end thereof distal to the distal end of the first energy-delivering member to sheathe the sharp distal tip of the first energy-delivering member. In some aspects, the first electrode portion and the second electrode portion have substantially the same outer diameters. In some aspects, the second insulation member is movable with respect to the second energy-delivering member. In some aspects, a window is defined in the second insulation member to expose varying lengths of the second electrode portion, and the distal end of the second insulation member is movable over the first electrode portion to expose varying lengths of the first electrode portion.

In accordance with various principles of the present disclosure, an energy-delivering treatment system includes an energy-delivering assembly having a first energy-delivering member having a sharp distal tip configured to puncture tissue, and a lumen defined therethrough and having a distal opening adjacent the sharp distal tip for delivery of materials distally through the lumen and out the distal opening thereof and/or for aspiration of materials into the distal opening of the lumen and proximally through the lumen; and a first insulation member extending over the first energy-delivering member and having a distal end. In some aspects, a first electrode portion of the energy-delivering assembly is defined along the first energy-delivering member between the sharp distal tip thereof and the distal end of the first insulation member; and a handle, the energy-delivering assembly extending distally from the handle. In some aspects, the handle includes a first control section operatively coupled with the first insulation member to adjust the position of the first insulation member with respect to the first energy-delivering member; and is configured to be operatively coupled with an energy source to supply energy to the first energy-delivering member.

In some aspects, the energy-delivering treatment system further includes a sheath extending distally from the handle, the first energy-delivering member and the first insulation member extending distally from the handle through the sheath; and a second control section operatively coupled with the first energy-delivering member to adjust a position thereof relative to the sheath.

In some aspects, the energy-delivering treatment system further includes a second energy-delivering member positioned over the first insulation member and having a distal end; a second insulation member extending over the second energy-delivering member and having a distal end; and a third control section operatively coupled with the second insulation member to adjust a position thereof relative to the second energy-delivering member. In some aspects, a second electrode portion of the energy-delivering assembly is defined along the second energy-delivering member between the distal end thereof and the distal end of the second insulation member.

In accordance with various principles of the present disclosure, a method of treating a target site within a human patient includes delivering energy to a first energy-delivering member covered by a first insulation member to define a first electrode portion at a distal end of the first energy-delivering member; and delivering materials through a lumen defined through the first energy-delivering member and with respect to the target site. In some aspects, delivering materials includes at least one of delivering materials distally through the lumen to the target site or aspirating materials proximally through the lumen from the target site.

In some aspects, the method further includes adjusting the length of the first electrode portion.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an elevational view of an example of an embodiment of an energy-delivering treatment system formed in accordance with aspects of the present disclosure.

FIG. 1A illustrates a detail view along detail area 1A in FIG. 1 illustrating further details of an example of an embodiment of an energy-delivering assembly formed in accordance with various principles of the present disclosure and usable in an energy-delivering treatment system as illustrated in FIG. 1.

FIG. 2 illustrates a cross-sectional view along line II-II in FIG. 1A.

FIG. 3 illustrates a cross-sectional view similar to FIG. 2, but illustrating another example of an embodiment of an energy-delivering assembly.

FIG. 4A illustrates an elevational view of another example of an embodiment of an energy-delivering treatment system formed in accordance with aspects of the present disclosure, positioned in a first configuration corresponding to a first configuration of the energy-delivering assembly thereof.

FIG. 4B illustrates an elevational view of the example of an embodiment of an energy-delivering treatment system illustrated in FIG. 4A, positioned in a second configuration corresponding to a second configuration of the energy-delivering assembly thereof.

FIG. 5 illustrates an example of an embodiment of an adjustable bipolar energy-delivering assembly formed in accordance with various principles of the present disclosure with the inner/first/distal energy-delivering member illustrated in an elevational view, and the insulation members and outer/second/proximal energy-delivering member illustrated in cross-section.

FIG. 6A illustrates a cross-sectional view of an example of an embodiment of an adjustable bipolar energy-delivering assembly with a first energy-delivering member illustrated in an elevational view and sheathed by a second energy-delivering member illustrated in cross-section.

FIG. 6B illustrates the adjustable bipolar energy-delivering assembly of FIG. 6A with the first energy-delivering member advanced distally out of the second energy-delivering member and into a target site.

FIG. 6C illustrates the adjustable bipolar energy-delivering assembly of FIG. 6A and FIG. 6B with the second energy-delivering member advanced into the target site.

FIG. 7A illustrates an elevational view of an example of an embodiment of an energy-delivering treatment system formed in accordance with various principles of the present disclosure and configured to adjust the example of an embodiment of an energy-delivering assembly illustrated in FIG. 6A.

FIG. 7B illustrates an elevational view of the energy-delivering treatment system illustrated in FIG. 7A in a configuration corresponding with the configuration of the energy-delivering assembly illustrated in FIG. 6B.

FIG. 7C illustrates an elevational view of the energy-delivering treatment system illustrated in FIG. 7A in a configuration corresponding with the configuration of the energy-delivering assembly illustrated in FIG. 6C.

FIG. 8A illustrates a cross-sectional view of an example of an embodiment of an adjustable bipolar energy-delivering assembly formed in accordance with various principles of the present disclosure, positioned in a monopolar configuration.

FIG. 8B illustrates the adjustable bipolar energy-delivering assembly illustrated in FIG. 8A, positioned in a bipolar configuration.

FIG. 9A illustrates a cross-sectional view of an example of an embodiment of an adjustable bipolar energy-delivering assembly formed in accordance with various principles of the present disclosure, with the proximal energy-delivering member in a first configuration.

FIG. 9B illustrates the adjustable bipolar energy-delivering assembly of FIG. 9A with the proximal energy-delivering member in a second configuration.

FIG. 10A illustrates a cross-sectional view of an example of an embodiment of an adjustable bipolar energy-delivering assembly formed in accordance with various principles of the present disclosure, with the energy-delivering members thereof in a first configuration.

FIG. 10B illustrates the bipolar adjustable energy-delivering assembly of FIG. 10A with the energy-delivering members thereof in a second configuration.

FIG. 10C illustrates an elevational view of the bipolar adjustable energy-delivering assembly of FIG. 10A.

FIG. 10D illustrates an elevational view of the bipolar adjustable energy-delivering assembly of FIG. 10C.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated.

Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As understood herein, corresponding is intended to convey a relationship between components, parts, elements, etc., configured to interact with or to have another intended relationship with one another. As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. For the sake of convenience, reference may be made to terms such as therapy, treatment, diagnosis, procedure, etc., including various grammatical forms thereof, alternately and without intent to limit, reference to one such term not excluding the others unless explicitly stated. Moreover, it will be appreciated that reference may be made herein to a treatment site, target site, site, etc., interchangeably and without intent to limit. Finally, reference to “at” a location or site is intended to include at and/or about the vicinity of (e.g., along, adjacent, proximate, etc.) such location or site.

As generally used herein, the term “ablation” generally refers to removal of cells either directly or indirectly by supply of energy within an electric field and may include removal by loss of cell function, cell lysis, coagulation, protein denaturation, necrosis, apoptosis, and/or irreversible electroporation. “Ablation” may similarly refer to creation of a lesion by ablation. Additionally, the terms “undesirable tissue,” “target cells,” “diseased tissue,” “diseased cells,” “tumor,” “cell mass” may be used herein to refer to cells removed or to be removed, in whole or in part, by ablation, and are not intended to limit application of any assemblies, systems, devices, or methods described herein. For example, such terms include ablation of both diseased cells and certain surrounding cells, despite no definite indication that such surrounding cells are diseased. Ablation performed by assemblies, systems, devices, or methods described herein may be of cells within tissue or located around a biological lumen (e.g., a vascular, ductal, or tract area). In accordance with various principles of the present disclosure, devices, assemblies, systems, and methods disclosed herein may be configured for performing ablation via electroporation and/or IRE.

In accordance with various principles of the present disclosure, an energy-delivering treatment system includes an energy-delivering assembly with a first energy-delivering member having an electrically-conductive elongate body defining an electrode portion therealong. More particularly, the electrically conductive elongate body may be formed of an electrically conductive material such as medical grade stainless steel, platinum, gold, nitinol, a cobalt-chromium alloy, a nickel-cobalt alloy such as MP35N, or other alloys, or materials plated with electrically-conductive materials, etc. An insulation member is positioned around a proximal portion of the first energy-delivering member, such as to insulate a proximal portion of the first energy-delivering member (to prevent delivery of energy to a patient along such insulated portion of the first energy-delivering member). A distal portion of the first energy-delivering member which is not positioned within/covered by the insulation member defines a first electrode portion of the energy-delivering assembly. The first electrode portion may be considered to be defined/to extend between the distal end of the first energy-delivering member and the distal end of the insulation member. There may or may not be insulation around other portions of the energy-delivering assembly. In accordance with various principles of the present disclosure, a lumen may be defined through the first energy-delivering member and is configured to deliver materials (e.g., treatment materials) distally therethrough and out the distal end thereof (e.g., adjacent/at the distal end of the first energy-delivering member) to the target site. Additionally or alternatively, the lumen may be configured to suction or aspirate materials from the target site into the distal end of the lumen (e.g., adjacent or at the distal end of the first energy-delivering member) proximally into and through the lumen. In some aspects, the distal end of the first energy-delivering member is configured as a sharp distal end which may be configured to pierce and/or puncture tissue, such as tissue at the target site. As such, in some aspects, the first energy-delivering member of an energy-delivering assembly formed in accordance with various principles of the present disclosure does not simply form an electrode portion of an energy-delivering assembly, but also may be a material transport device with a lumen capable of delivering materials (e.g., treatment/therapeutic materials, irrigation materials, chemotherapy agents, immunogenic agents, gel isolation materials, embolic materials, etc.), and/or other devices (e.g., guidewires, fiducial markers, sensing devices (e.g., psi, pH, temperature, etc.), imaging devices, etc.) to the target site; and/or suctioning/aspirating materials (fluids, tissue samples, etc.) from the target site.

The energy-delivering assembly may be deliverable through an elongate tubular member (e.g., a delivery sheath, catheter, working channel of an endoscope, etc.) inserted into a patient (such as through a natural anatomical passage or orifice and into a body lumen within a patient), or transcutaneously or percutaneously or even surgically. The energy-delivering member of the energy-delivering assembly may be coupled to an energy source to energize the electrode portion to apply an electric current to biological tissue. The energy source may be operative to generate an electric field between the electrode portion and another electrode portion, such as an electrode portion coupled to the energy source and having an opposite polarity, e.g., a return or ground.

An energy-delivering assembly formed in accordance with various principles of the present disclosure may be a monopolar device inserted into the patient with a return or grounding electrode positioned on the exterior of the patient (in contrast with within the patient, such as on the epidermis of the patient). Alternatively, the energy-delivering assembly may be a bipolar device with at least two electrodes configured to be placed and positioned at or near undesirable tissue (e.g., target cells, target site, treatment site, diseased tissue, diseased cells, tumor, cell mass) in a tissue treatment region (e.g., a target region or target site) within the patient's body. Depending on the desired application, for example, the diagnostic or therapeutic treatment to be performed, a particular electrode may be configured either as an anode or a cathode, or a plurality of electrodes may be configured with at least one electrode configured as an anode and at least one other electrode configured as a cathode.

Once an energy-delivering assembly formed in accordance with various principles of the present disclosure is positioned at or near undesirable tissue, an energizing potential may be applied to the electrode portions thereof, such as to create an electric field to which the tissue at the target site is exposed. The energizing potential (and the resulting electric field) may be characterized by various parameters, such as, for example, frequency, amplitude, pulse width (duration of a pulse or pulse length). Suitable energy sources include electrical waveform generators, such as waveform generators capable of creating IRE, high frequency IRE, NanoPulse, and/or ablative waveforms. The energy source generates an electric field with desired characteristics for the treatment to be performed at the target site. For instance, the electric field may be generated to have suitable characteristic waveform output in terms of voltage, impedance, frequency, amplitude, pulse width, delays between pulses, number of pulses per burst, number of bursts, and polarity. The electric current flows between the electrodes and through the tissue proportionally to the potential (e.g., voltage) applied to the electrodes. The supplied electric current provided by the energy source may deliver a pulse sequence to the target site. For example, an energy source may supply various waveforms in one or more pulse sequences tailored to the desired application.

In accordance with various principles of the present disclosure, an electrode of an energy-delivering assembly formed in accordance with various principles of the present disclosure (the electrode of a monopolar energy-delivering assembly, or one or both electrodes of a bipolar energy-delivering assembly) may be adjustable to vary the electric field generated along the electrode portion(s) thereof. For instance, it may be desirable to adjust various characteristics of the electrode and the energy field generated therearound based on the characteristics of the target site (e.g., the size of a tumor to be treated, and/or variations along the target site, etc.), such as may be determined by pre-diagnostic, CT, MRI, endoscopic, and/or ultrasound or other imaging technique known those of ordinary skill in the art, typically before the energy-delivering treatment system has been delivered to the target site. It may also be adjusted again once the energy-delivering treatment system has been delivered to the target site, based upon ultrasound or other imaging. In accordance with various principles of the present disclosure, the length of an electrode portion of an energy-delivering assembly may be adjustable, such as to adjust the length and/or volume of the energy field generated therearound upon application of energy thereto. Optionally, the voltage may be adjusted to further adjust the energy field (e.g., to adjust primarily the radial diameter of the energy field).

In accordance with various principles of the present disclosure, a monopolar energy-delivering assembly includes a first energy-delivering member. The energy-delivering member optionally has a lumen therethrough such that the first energy-delivering member not only delivers energy treatment/therapy to a target site, but also may deliver materials to and/or remove materials from a target site. In accordance with further various principles of the present disclosure, the first energy-delivering member may have a sharp distal tip and optionally the distal end region of the energy-delivering member is configured to facilitate smooth puncture/entry of the energy-delivering member into the target site. Additionally or alternatively, in accordance with various further principles of the present disclosure, an energy-delivering member of an energy-delivering assembly is adjustable, such as adjustable in length.

In some aspects, an energy-delivering assembly formed in accordance with various principles of the present disclosure is a bipolar energy-delivering assembly. In accordance with various principles of the present disclosure, the bipolar energy-delivering assembly includes a first energy-delivering member and a second energy-delivering member. In accordance with various principles of the present disclosure, the first energy-delivering member optionally has a lumen defined therethrough, such as described above with reference to a monopolar energy-delivering assembly. In some aspects, one or both of the first and second energy-delivering members are adjustable and/or are adjustable with respect to each other. Additionally or alternatively, a distal portion of a bipolar energy-delivering assembly is configured to facilitate smooth puncture/entry of the energy-delivering member into the target site.

Energy-delivering assemblies, devices, systems, and methods described herein may be utilized for electroporation, irreversible electroporation (IRE), and/or electropermeabilization techniques to apply external electric fields (electric potentials) to cell membranes to significantly increase permeability of the plasma membrane of the cell, such as to improve uptake of therapeutic materials by the cell. Optionally, the energy applied to the cell may change the characteristics of the cell membranes (e.g., porosity), such as irreversibly, resulting in cell death (e.g., by apoptosis and/or necrosis). Such techniques may advantageously be used to treat/apply therapy without raising the temperature of the surrounding tissue to a level at which permanent damage may occur to the surrounding tissue, support structure, and/or regional vasculature Application of IRE pulses to cells may thus be an effective way for ablating large volumes of undesirable tissue with no or minimal detrimental thermal effects to the surrounding healthy tissue. This device can also be used for radiofrequency ablation, which causes thermal coagulation of the tissue.

Various embodiments of electrode devices, assemblies, systems, and various associated methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

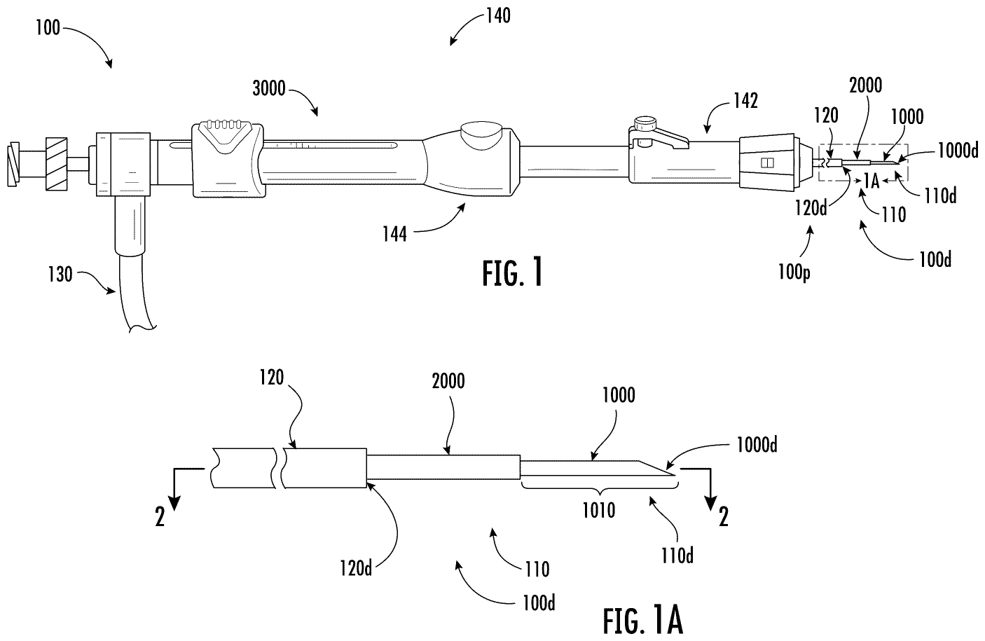

Turning now to the drawings, an example of an embodiment of an energy-delivering treatment system 100 is illustrated in FIG. 1. An energy-delivering assembly 110 and, optionally, a sheath 120 (extendable over the energy-delivering assembly 110) which extend from the distal end 100d of the energy-delivering treatment system 100. The energy-delivering assembly 110 includes an energy-delivering member 1000, and an insulation member 2000 extending over at least a proximal portion of the energy-delivering member 1000. The energy-delivering member 1000 and its insulation 2000 are typically configured to be selectively distally extendable out the distal end 120d of the sheath 120, such as to protect the passage through which the energy-delivering assembly 110 is extended (e.g., a working channel of an endoscope, a body lumen, etc.) from a sharp distal end 1000d of the energy-delivering member 1000 (described in further detail below), and proximally retractable to expose at least a distal portion of the energy-delivering assembly 110. Optionally, an energy source is coupled to the proximal end 100p of energy-delivering treatment system 100, such as via a power connector 130. The power connector 130 may include a plug and/or cable and/or other wiring configured to be coupled to an energy source (via a power delivery cable 132) such as known those of ordinary skill in the art and selectable by known means based on the type of energy to be applied by the energy-delivering treatment system 100.

The energy-delivering treatment system 100 optionally includes a handle 140 operatively coupled with the energy-delivering assembly 110, such as to control elements of the energy-delivering assembly 110, such as to control the position of the energy-delivering member 1000. The handle 140 may also be operatively coupled with the sheath 120 such as to control the position of the sheath 120 with respect to the energy-delivering assembly 110, such as in a manner known to those of ordinary skill in the art. In some embodiments, the handle 140 includes a first control section 142 operatively coupled with the sheath 120, such as coupled with and configured to adjust/control the position of the sheath 120 with respect to a delivery device through which the energy-delivering assembly 110 and sheath 120 are advanceable to a treatment site. It will be appreciated that the delivery device may be any desired delivery device, such as a delivery sheath or an endoscope, having a lumen or working channel therethrough sized to allow passage of the energy-delivering assembly 110 and sheath 120 therethrough, the present disclosure not being limited in this regard. The handle 140 includes a second control section 144 operatively coupled with the energy-delivering assembly 110, such as coupled with and configured to adjust/control the position of the distal end 110d of the energy-delivering assembly 110 with respect to the sheath 120. For instance, once the distal end 120d of the sheath 120 has been positioned at or adjacent a treatment site (e.g., by manipulation, control, etc. of the first control section 142), the energy-delivering assembly 110 may be extended distally beyond the distal end 120d of the sheath 120 by manipulation, control, etc., of the second control section 144.

In accordance with various principles of the present disclosure, the energy-delivering assembly 110 is an elongate flexible assembly capable of being navigated through a patient's body, such as through natural orifices and/or through tubular elongate members inserted into the patient's body. More particularly, the energy-delivering member 1000 of the energy-delivering assembly 110 may be elongate and sufficiently flexible to be able to be inserted transluminally into the body (e.g., endoscopically, such as in contrast with being percutaneously inserted) and navigated through potentially tortuous pathways within the body, or at least being capable of bending or turning with/within natural, nonlinear anatomical structures. Additionally or alternatively, the energy-delivering member 1000 may be sufficiently resilient so as not to break as it is being navigated. It will be appreciated that those of ordinary skill in the art may determine appropriate length, flexibility, resiliency, and/or other properties/characteristics of an energy-delivering member 1000 used in accordance with various principles of the present disclosure based on the material, size, shape, configuration, and/or dimensions of the energy-delivering member 1000, the present disclosure not necessarily being limited to specific parameters. In some aspects, an energy-delivering assembly 110 may be formed in accordance with various principles of the present disclosure with one or more components, elements, members, etc., which are adjustable in size, shape, configuration, and/or dimensions (such as relative to other components, elements, members, etc., of the energy-delivering assembly 110), as described in further detail below.

In some aspects, the energy-delivering member 1000 of the energy-delivering assembly 110 is a trocar (solid or with a lumen therethrough), needle, tip, tissue piercing device/electrode, able to easily pierce tissue/organs/tumor masses, such with or without coring tissue. The energy-delivering member 1000 may be any of a variety of needle types, including, without limitation, lancet, Franzene, Trocar, Sprotte, Pencil-Point, Chiba, Turner, Madayag, Greene, Menghini, Westcott, etc. In some aspects, the energy-delivering member 1000 can have a fine needle aspiration (FNA) or fine needle biopsy (FNB) tip configuration. In some aspects, the energy-delivering member 1000 has a flexibility selected to accommodate case of use with various delivery devices (such as delivery devices configured for transluminal delivery), and/or to have the ability to retract and/or deploy the energy-delivering member 1000 to a desired location in a tortuous anatomical structure without having unacceptable shape setting. In some aspects, the energy-delivering member 1000 may be as small as a 34 Ga needle, or up to a 6 Ga needle, including incremental sizes therebetween. The energy-delivering member 1000 may be any appropriate length for inserting into a patient to reach a desired target site.

In accordance with various principles of the present disclosure, the needle need not simply have a sharp distal end and/or a lumen defined therethrough, but may also be capable of being energized to deliver energy to a treatment site for therapeutic, diagnostic, treatment, etc., purposes. The voltage, current (e.g., waveform, pulse pattern, size and nature of energy pulse packets, frequency, etc.), power, and other parameters are adjusted as needed or indicated for the particular therapy or treatment or procedure being performed, the present disclosure not being limited by such details. The power connector 130 is electrically coupled with the energy-delivering member 1000 to supply the desired energy form to the energy-delivering member 1000. And, the energy-delivering member 1000 may be formed of an electrically conductive material (e.g., medical grade/biocompatible stainless steel, platinum, gold, nitinol, a cobalt-chromium alloy, a nickel-cobalt alloy such as MP35N, or other alloys, or materials plated with electrically-conductive materials), such as capable of establishing tissue contact/conductivity in order to deliver sufficient energy to ablate tissue and/or to apply sufficient energy to achieve ablation, and/or irreversible electroporation of tissue in the electric field of the energy-delivering member 1000.

As illustrated in the detail view of FIG. 1A, along detail 1A of FIG. 1, the illustrated example of an embodiment of an energy-delivering member 1000 formed in accordance with various principles of the present disclosure has an elongate body 1002 ending at a distal end 1002d which optionally includes a sharp distal tip 1004. It will be appreciated that the distal end 1002d of the energy-delivering member elongate body 1002 may be coextensive with the distal end 1000d of the energy-delivering member 1000, and reference may be made to the distal end 1000d or the distal end 1002d interchangeably, without intent to limit unless explicitly stated. Further in accordance with various principles of the present disclosure, a lumen 1003 is defined through the energy-delivering member 1000 and ends at a distal opening adjacent/at the sharp distal tip 1004 of the elongate body 1002.

Additionally or alternatively, the example of an embodiment of an insulation member 2000 illustrated in FIG. 1A and formed in accordance with various principles of the present disclosure has an elongate body 2002 defining a lumen 2003 through which the energy-delivering member 1000 extends. It will be appreciated that the distal end 2002d of the insulation member elongate body 2002 may be coextensive with the distal end 2000d of the insulation member 2000, and reference may be made to the distal end 2000d or the distal end 2002d interchangeably, without intent to limit unless explicitly stated.

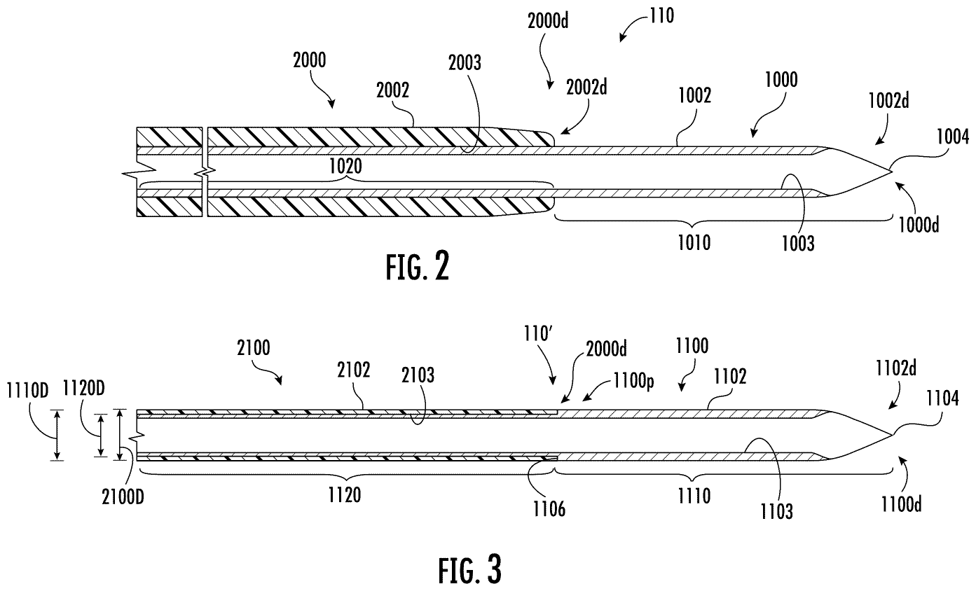

As illustrated in FIG. 1A, and the cross-sectional view illustrated in FIG. 2, taken along line II-II of FIG. 1A, a distal portion of the energy-delivering member 1000 extends distally beyond the distal end 2002d of the elongate body 2002 of the insulation member 2000 to define an electrode portion 1010 of the energy-delivering assembly 110 distal to the distal end 2000d of the insulation member 2000. More particularly, the electrode portion 1010 is defined along a distal longitudinal extent of the energy-delivering member 1000 between the distal end 1002d of the elongate body 1002 thereof and the distal end 2002d of the elongate body 2002 of the insulation member 2000 which is positioned over a proximal portion of the elongate body 1002 of the energy-delivering member 1000. The insulation member 2000 is formed of an insulative material (e.g., a polymer extrusion, a polymer heat shrink, non-metallic braided shaft, composite shaft, etc.) so that only the portion of the energy-delivering member 1000 extending beyond the distal end 2002d of the elongate body 2002 of the insulation member 2000 functions as an electrode (with proximal portions of the elongate body 1002 of the energy-delivering member 1000 within the insulation member 2000 insulated/shielded and thus not applying/transmitting energy to a patient). As such, the distance between the distal end 1002d of the energy-delivering member elongate body 1002 and the distal end 2002d of the insulation member elongate body 2002 defines the length of the electrode portion 1010 of the energy-delivering assembly 110. The proximal portion 1020 of the energy-delivering member elongate body 1002, proximal to the electrode portion 1010, is insulated by the insulation member 2000 and thus may be considered to simply function as an insulated needle without electrically therapeutic functions other than conducting/conveying energy to the electrode portion 1010 at the distal end 1002d of the energy-delivering member elongate body 1002.

As may be appreciated, in accordance with various principles of the present disclosure, the example of an embodiment of an energy-delivering assembly 110 illustrated in FIG. 2 provides a unique structure for delivering therapeutic energy to a patient. Such structure may be considered a monopolar needle, such as a monopolar IRE needle. Not only does the energy-delivering assembly 110 have an electrode area (which may also be referenced as a probe) for providing or delivering energy, but the energy-delivering assembly 110 may also have a lumen for delivery of materials (e.g., medicines, treatments, therapies, or irrigation fluids such as saline) or for aspiration of materials (e.g., tissue samples, or fluids or other materials, such as which may need to be cleared from the treatment site). For instance, the distal end 1002d of the energy-delivering member elongate body 1002 may be inserted into a lesion, and drugs, such as chemotherapy, may be applied before, after, or during the time of treatment (e.g., in conjunction with the energy treatment being applied by the electrode portion 1010 of the energy-delivering assembly 110). Because IRE allows for pores to open in cells, delivery of treatment materials by the electroporation electrode, such as achievable by the energy-delivering assembly 110 of the present disclosure, may serve a synergistic function, allowing the chemotherapy to be more readily taken in by the cells, thereby increasing the effectiveness of the chemotherapy.

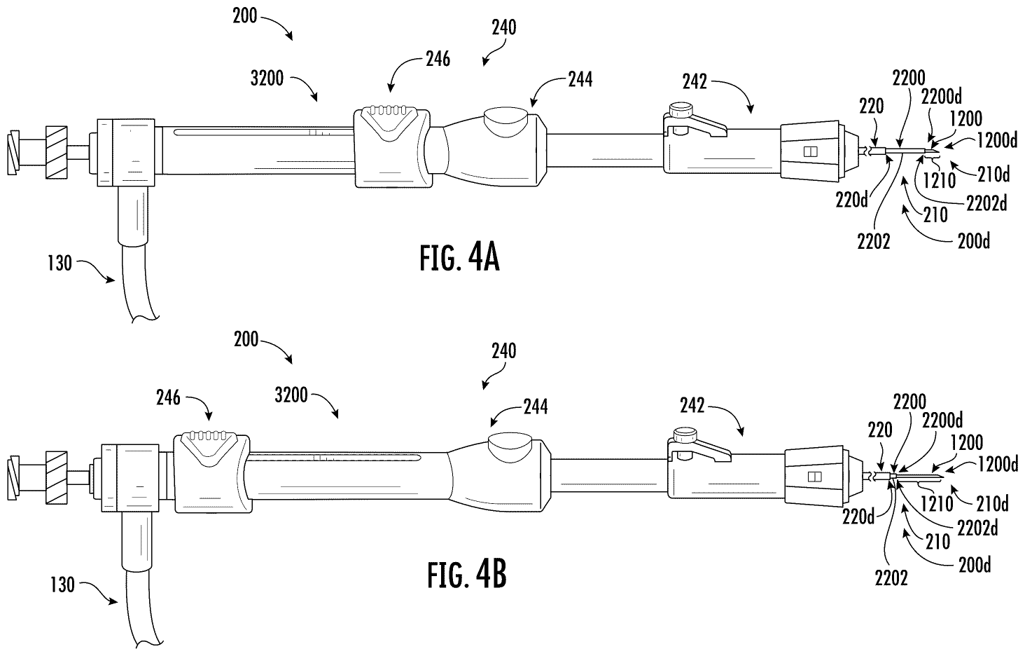

In the example of an embodiment illustrated in FIG. 2, the energy-delivering member 1000 may have a substantially constant outer diameter, with the insulation member 2000 formed or otherwise positioned thereover having an outer diameter larger than the outer diameter of the energy-delivering member 1000. Alternatively, the example of an embodiment of an energy-delivering assembly 110′ illustrated in FIG. 3 has an energy-delivering member 1100 with a proximal portion 1120 stepped, at a shoulder 1106, with respect to the electrode portion 1110 defined along a distal portion of the energy-delivering member 1100. In some aspects, the proximal portion 1120 of the energy-delivering member 1100 has a reduced outer diameter 1120D relative to the outer diameter 1110D of the electrode portion 1110 defined at the distal end 1102d of the energy-delivering member elongate body 1102. Such reduced outer diameter 1120D provides a space around the energy-delivering member elongate body 1102 for the insulation member 2100 to fit therearound (with the proximal portion 1120 of the energy-delivering member 1100 fitting within a lumen 2103 defined through the insulation member 2100), such as illustrated in FIG. 3. In some embodiments, the outer diameter 2100D of the insulation member 2100 may be substantially equal to (and thus substantially flush with) the outer diameter 1110D of the electrode portion 1110. Such configuration may facilitate a smoother puncture of tissue by the sharp distal tip 1104 of the energy-delivering member elongate body 1102. It will be appreciated that various elements of the energy-delivering assembly 110′ illustrated in FIG. 3 are referenced with the same reference elements as in FIG. 2, increased by 100 to indicate similar or same structures or features, and redundant description is generally omitted for the sake of brevity. Moreover, the example of an embodiment of an energy-delivering assembly 110′ illustrated in FIG. 3 may be used with the various components of the example of an embodiment of an energy-delivering treatment system 100 illustrated in FIG. 1, as may be appreciated by one of ordinary skill in the art.

Various modifications to the basic structure, characteristics, features, properties, etc., of an energy-delivering member and/or an insulation member of an energy treatment assembly of the present disclosure may be made in accordance with various principles of the present disclosure. For instance, the number of energy delivering members and/or insulation members; the positioning of the energy delivering member(s) and/or insulation member(s) with respect to one another; the manner of coupling the energy delivering member(s) and/or insulation member(s); the size, shape, configuration, and/or dimensions of the energy delivering member(s) and/or insulation member(s); and other structure, characteristics, features, properties, etc., of the energy delivering member(s) and/or insulation member(s) may be varied to form an energy treatment assembly in accordance with various principles of the present disclosure, as will now be described with reference to the various further figures accompanying the present disclosure.

It may be desirable to allow adjustability of various characteristics of the energy-delivering portion (e.g., the electrode portion) of a monopolar needle, such as to adjust for variations in size, shape, etc., of the treatment site. For instance, longer electrodes generally create longer ablation volumes, which may be needed depending on the nature and characteristics of the treatment site. Additionally or alternatively, to further increase the radial diameter of the ablated region, the applied voltage can be increased. In accordance with various principles of the present disclosure, in some aspects, an energy-delivering assembly as described herein may be adjustable in size, shape, configuration, and/or dimensions.

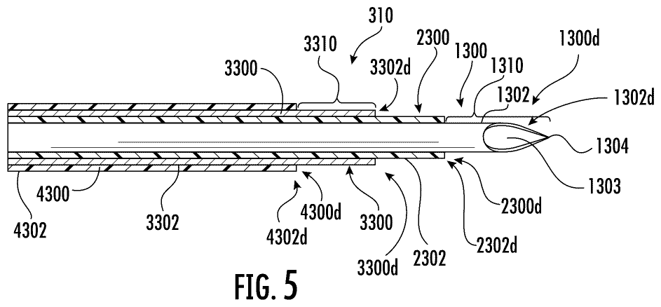

An example of an embodiment of an energy-delivering treatment system 200 with an adjustable (e.g., adjustable length) monopolar energy-delivering assembly 210 formed in accordance with various principles of the present disclosure is illustrated in FIG. 4A and FIG. 4B. It will be appreciated that various elements of the example of an embodiment of an energy-delivering treatment system 200 illustrated in FIG. 4A and FIG. 4B are referenced with the same reference elements as in FIG. 1 and FIG. 2, increased by 100 to indicate similar or same structures or features, and redundant description is generally omitted for the sake of brevity.

Similar to the example of an embodiment illustrated in FIG. 2, the energy-delivering member 1200 has an electrode portion 1210 defined by the portion of the energy-delivering member 1200 extending distal to the distal end 2200d of the insulation member 2200 (between the energy-delivering member end 1200d/1202d and the insulation member end 2200d/2202d). However, as may be appreciated upon comparing the energy-delivering assembly 210 extending from the distal end 200d of the energy-delivering treatment system 200 illustrated in FIG. 4A and FIG. 4B, the energy-delivering member 1200 is movable with respect to (e.g., slidable proximally within or distally out of) the insulation member 2200 to vary the exposed length thereof which forms the electrode portion 1210. More particularly, in the example of an embodiment illustrated in FIG. 4A, the distal end 2200d of the insulation member 2200 is positioned a first distance from (proximal to) the distal end 1202d of the energy-delivering member 1200. And, in the example of an embodiment illustrated in FIG. 4B, the distal end 2200d of the insulation member 2200 is positioned (after the energy-delivering member 1100 and the insulation member 2200 are moved relative to each other) a second distance from (proximal to) the distal end 1202d of the energy-delivering member 1200, the second distance being greater than the first distance. As such, the electrode portion 1210, has a first length corresponding to the first distance in FIG. 4A, and a second length corresponding to the second distance in FIG. 4B, the second length being greater than the first length. The variation in length of the electrode portion 1210 may be achieved by relative movement of the energy-delivering member 1200 and the insulation member 2300 such as by proximally retracting the insulation member 2300 relative to the energy-delivering member 1200, and/or distally extending the energy-delivering member 1200 relative to the insulation member 2200. It will be appreciated that distances between the distal end 1200d of the energy-delivering member 1200 (and the distal end 1202d of the elongate body 1202 thereof) and the distal end 1200d of the insulation member 2200 (and the distal end 2202d of the elongate body 2202 thereof), and thus the lengths of the electrode portion 1210 defined between such ends 1200d, 2200d, may be variable with relatively infinite adjustability, or adjustable in pre-fixed increments, upon relative movement (e.g., sliding) of the energy-delivering member 1200 and the insulation member 2200. As may be appreciated, the energy-delivering member 1200 and the insulation member 2200 may be distally advanceable out of, or proximally retractable into a sheath 220 in a manner as described above with respect to the example of an embodiment of a sheath 120 illustrated FIG. 1, reference being made thereto for further applicable descriptions as may be appreciated by those of ordinary skill in the art.

In some aspects, to facilitate adjustment of the energy-delivering assembly 210 of the example of an embodiment of an energy-delivering treatment system 200 as illustrated in FIG. 4A and FIG. 4B, a handle 240 may be operatively coupled with one or more elements of the energy-delivering assembly 210 to control movement thereof. More particularly, in accordance with various principles of the present disclosure, the example of an embodiment of an energy-delivering treatment system 200 illustrated in FIG. 4A and FIG. 4B includes a handle 240 with a first control section 242, a second control section 244, and a third control section 246. The first control section 242 is operatively coupled with the sheath 220 and may be substantially similar (e.g., in form and/or function) to the first control section 142 described above with reference to FIG. 1, reference being made to the description thereof for the sake of brevity and without intent to limit. The second control section 244 is operatively coupled with the energy-delivering member 1200 of the energy-delivering assembly 110, such as coupled with and configured to control the position of the distal end 1200d of the energy-delivering member 1200 with respect to the sheath 220. The second control section 244 may be substantially similar (e.g., in form and/or function) to the second control section 144 described above with reference to FIG. 1, reference being made to the description thereof for the sake of brevity and without intent to limit. The third control section 246 of the example of an embodiment of a handle 240 illustrated in FIG. 4A and FIG. 4B is operatively coupled with the insulation member 2200, such as coupled with and configured to control the position of the distal end 2202d of the insulation member 2200 with respect to the distal end 1200d energy-delivering member 1200. In some aspects, the insulation member 2200 is slidable with respect to the energy-delivering member 1200, such as upon sliding or other movement of the third control section 246. For instance, the third control section 246 may be movable proximally (e.g., from the position illustrated in FIG. 4A to the position illustrated in FIG. 4B) to proximally retract the insulation member 2200 with respect to the energy-delivering member 1200 to increase the length of the electrode portion 1210 of the energy-delivering member 1200. Optionally, to reduce friction between the insulation member 2200 and the energy-delivering member 1200, the insulation member 2200 may include an inner tubular element formed of a material facilitating movement with respect to the energy-delivering member 1200. For instance, in some embodiments, the insulation member 2200 may be formed of a metal hypotube covered with an insulative material such as a polymeric insulative material (e.g., extrusion, heat shrink, etc.). Other configurations are within the scope and spirit of the present disclosure, the present disclosure not being limited in this regard.

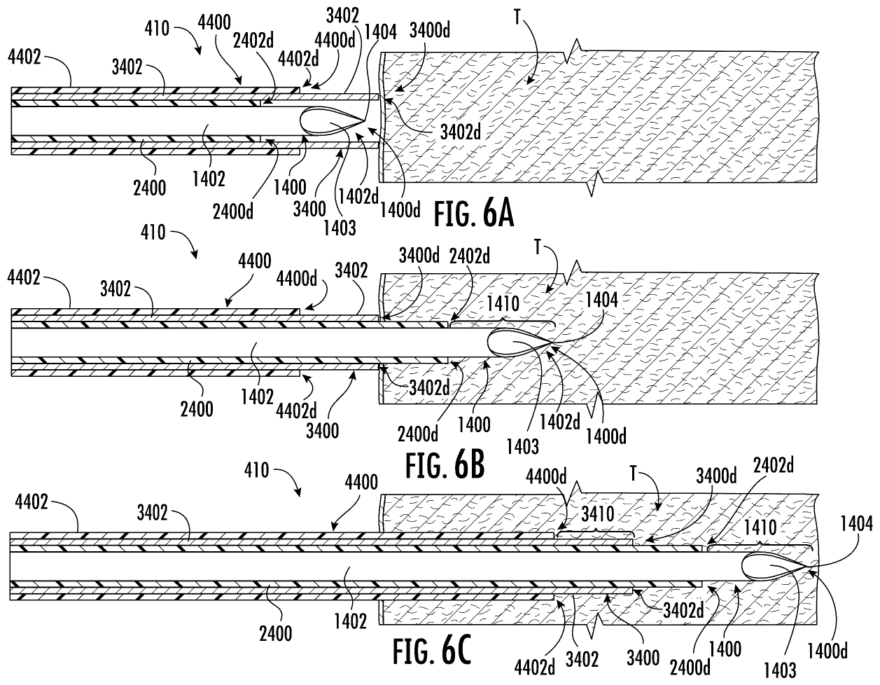

In accordance with various principles of the present disclosure, various of the above-described principles may be applied to form a bipolar, rather than a monopolar, adjustable energy-delivering assembly 310, such as with a bipolar needle probe, such as illustrated in FIG. 5. For instance, the example of an embodiment of an adjustable bipolar energy-delivering assembly 310 illustrated in FIG. 5 has a first energy-delivering member 1300 over which a first insulation member 2300 is positioned. The first energy-delivering member 1300 and the first insulation member 2300 may be substantially similar to the energy-delivering member 1200 and the insulation member 2200 described above with reference to the example of an embodiment of an adjustable monopolar energy-delivering assembly 210 illustrated in FIG. 4A and FIG. 4B. Accordingly, for the sake of brevity, and without intent to limit, the same reference characters used in FIG. 4A and FIG. 4B, increase by 100, are used with respect to same or similar elements illustrated in FIG. 5, and, for the sake of brevity, redundant descriptions are omitted (reference being made to the above-provided descriptions as applicable to the same or similar elements in FIG. 5). However, in contrast with the adjustable energy-delivering assembly 210 illustrated in FIG. 4A and FIG. 4B, the adjustable energy-delivering assembly 310 illustrated in FIG. 5 has a second electrode which may, like the first electrode, be insulated. More specifically, the adjustable bipolar energy-delivering assembly 310 illustrated in FIG. 5 includes a second energy-delivering member 3300 and a second insulation member 4300 extending over at least a proximal portion of the second energy-delivering elongate member 3300. The second energy-delivering member 3300 may be an electrically conductive tubular member through which the first energy-delivering member 1300 and the first insulation member 2300 are distally advanceable and/or proximally retractable. In some embodiments, the second energy-delivering member 3300 is a conductive hypotube, such as a metal (e.g., medical grade/biocompatible stainless steel, platinum, gold, nitinol, a cobalt-chromium alloy, a nickel-cobalt alloy such as MP35N, or other alloys, or materials plated with electrically-conductive materials) hypotube, and may be formed of a material which is the same or similar to or different from the material from which the first energy-delivering member 1300 is formed. The second insulation member 4300 may be similar or substantially the same as or different from the first insulation member 2300 other than dimensions (e.g., the second insulation member 4300 is larger than the first insulation member 2300 to allow the first energy-delivering member 1300, the first insulation member 2300, and the second energy-delivering member 3300 to extend therethrough). Although the first insulation member 2300 and the second insulation member 4300 may be substantially the same, they need not be, as each insulation member 2300, 4300 may have a somewhat different purpose and need for the energy-delivering assembly 310 to function correctly and safely. In some aspects, the second energy-delivering member 3300 supports the insulation member 4300 positioned therearound. It will be appreciated that, in a practical sense, the second energy-delivering member 3300 should have a full layer of insulation, such as provided by the first insulation member 2300 (whether adjustable or not), between itself and the first energy-delivering member 1300, as well as a layer of insulation, such as provided by the second insulation member 4300 (whether adjustable or not). Like the energy-delivering assembly 210 illustrated in FIG. 4A and FIG. 4B, a sheath (not illustrated) may be extendable over the energy-delivering members 1300, 3300 and insulation members 2300, 4300 of the energy-delivering assembly 310 illustrated in FIG. 5. Such sheath may be substantially the same in nature and characteristics of the above-described sheath 120, reference therefore being made to such description as applicable to a sheath usable with the energy-delivering assembly 310 illustrated in FIG. 5.

The energy-delivering members 1300, 3300 of the example of an embodiment of an adjustable bipolar energy-delivering assembly 310 illustrated in FIG. 5 provide a needle electrode/probe with similar benefits as described with respect to the above-described monopolar energy-delivering assemblies 110, 110′, 210, allowing energy-based treatment along with a material delivery based treatment and/or material sampling and/or aspirating. Additionally, like the above-described adjustable monopolar energy-delivering assembly 210, the adjustable bipolar energy-delivering assembly 310 illustrated in FIG. 5 allows for adjustability (e.g., in length) of the energy-delivering member thereof. However, the energy-delivering assembly 310 illustrated in FIG. 5 includes two energy-delivering members 1300, 3300, and allows for adjustability of a first energy-delivering member 1300 as well as optional adjustability of a second energy-delivering member 3300. Moreover, the energy-delivering assembly 310 illustrated in FIG. 5 allows for adjustability of the insulation member 2300 between the first energy-delivering member 1300 and the second energy-delivering member 3300, such as to adjust the distance of the two electrodes of the bipolar needle probe/electrode of the energy-delivering assembly 310, and therefore adjusting the active surface area of the energy-delivering assembly 310. In particular, similar to the adjustable monopolar energy-delivering assembly 210 illustrated in FIG. 4A and FIG. 4B, the first energy-delivering member 1300 of the energy-delivering assembly 310 defines a first electrode portion 1310 between the distal end 1300d of the first energy-delivering member 1300 (e.g., the distal end 1302d of the elongate body 1302 of the first energy-delivering member 1300) and the distal end 2300d of the first insulation member 2300 (e.g., the distal end 2302d of the elongate body 2302 of the first insulation member 2300). Further similar to the adjustable monopolar energy-delivering assembly 210 illustrated in FIG. 4A and FIG. 4B, the first energy-delivering member 1300 and the first insulation member 2300 are movable with respect to each other to adjust the length of the electrode portion 1310 of the energy-delivering member 1300 defined between the energy-delivering member end 1300d/1302d and the insulation member end 2200d/2202d.

The second energy-delivering member 3300 of the example of an embodiment of an adjustable bipolar energy-delivering assembly 310 illustrated in FIG. 5 defines a second electrode portion 3310 of the energy-delivering assembly 310 between the distal end 3300d of the second energy-delivering member 3300 (e.g., the distal end 3302d of the elongate body 3302 of the second energy-delivering member 3300) and the distal end 4300d of the second insulation member 4300 (e.g., the distal end 4302d of the elongate body 4302 of the second insulation member 4300). Like the first energy-delivering member 1300, the second energy-delivering member 3300 defines a second electrode portion 3310 between the distal end 3300d of the second energy-delivering member 3300 (e.g., the distal end 3302d of the elongate body 3302 of the second energy-delivering member 3300) and the distal end 4300d of the second insulation member 4300 (e.g., the distal end 4302d of the elongate body 4302 of the second insulation member 4300). Furthermore, similar to the first energy-delivering member 1300 and the first insulation member 2300, the second energy-delivering member 3300 and the second insulation member 4300 are movable with respect to each other to adjust the length of the second electrode portion 3310 of the second energy-delivering member 3300 defined between the second energy-delivering member distal end 3300d/3302d and the second insulation member distal end 4200d/4202d.

In use, energy (e.g., electrical current) is delivered to the energy-delivering assembly 310 to pass to and between the first electrode portion 1310 and the second electrode portion 3310 of the energy-delivering assembly 310. As may be appreciated, one of the first electrode portion 1310 and the second electrode portion 3310 serves as an anode of the bipolar energy-delivering assembly 310 and the other of the first electrode portion 1310 and the second electrode portion 3310 serves as a cathode of the bipolar energy-delivering assembly 310. Any or each of the first energy-delivering member 1300, the first insulation member 2300, the second energy-delivering member 3300, and the second insulation member 4300 may be movable with respect to one another to vary the lengths, distances, etc., of the electrode portions 1310, 3310 and/or the insulation members 2000, 4300 (e.g., to vary the distance between the distal end of the proximal, second energy-delivering member 3300 and the proximal end of the distal, first energy-delivering member 1300 adjacent the distal end 2300d of the first insulation member 2300) and the associated and other relevant properties and characteristics of the energy-delivering assembly 310 as may be appreciated by those of ordinary skill in the art. Moreover, any or each of the first energy-delivering member 1300, the first insulation member 2300, the second energy-delivering member 3300, and the second insulation member 4300 may be operatively coupled with an associated handle component movable (e.g., slidable) to distally retract or axially advance/extend the associated member 1300, 2300, 3300, 4300 operatively coupled therewith. A handle and associated components as described above may be operatively coupled with the example of an embodiment of an energy-delivering assembly 310 illustrated in FIG. 5.

It will be appreciated that in order to readily be navigated within a patient's body, the members 1300, 2300, 3300, 4300 of the energy-delivering assembly 310 illustrated in FIG. 5 typically must be thin enough to result in an appropriate overall size and flexibility for navigation, delivery, and use of the energy-delivering assembly 310. Moreover, as discussed above, standard fine needle aspiration (FNA) devices have a needle contained within a protective sheath that protects the working channel of the delivery device (e.g., an endoscope) used to deliver the energy-delivering assembly and/or the patient's tissue from being punctured by the needle tip. For instance, as described with reference to FIG. 1, the energy-delivering assembly 110 may be delivered to the target site within a sheath 120 with the sharp distal tip 1004 of the energy-delivering member 1000 proximal to a distal end 120d of the sheath 120. Once the protective sheath is passed through the delivery device and extends out the distal end of the delivery device, the FNA needle is advanced out the distal end of this sheath during puncture, and the sheath generally does not enter the tissue. A similar sheath may be used with the example of an embodiment of an energy-delivering assembly 310 illustrated in FIG. 5.

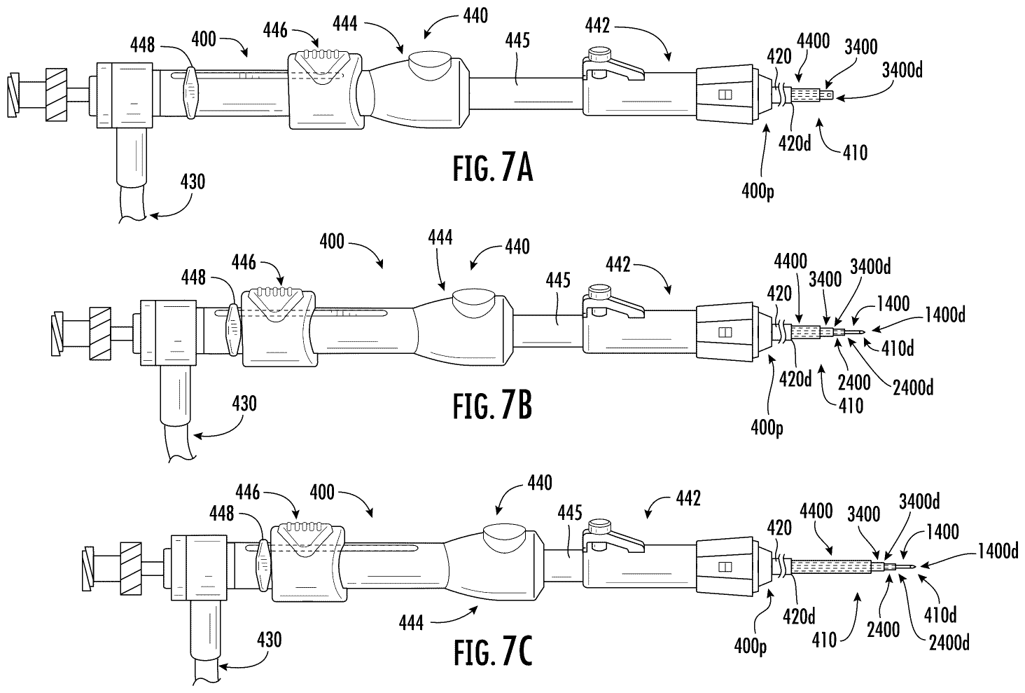

In accordance with various principles of the present disclosure, as illustrated in FIG. 6A, FIG. 6B, and FIG. 6C, an adjustable bipolar energy-delivering assembly 410 may be formed with a proximal, second energy-delivering member 3400 forming a protective sheath for the distal, first energy-delivering member 1400 which is in the form of a needle, such as an FNA needle. More particularly, the example of an embodiment of an adjustable bipolar energy-delivering assembly 410 illustrated in FIG. 6A, FIG. 6B, and FIG. 6C has a first energy-delivering member 1400 with a first insulation member 2400 positioned therearound. The first energy-delivering member 1400 and the first insulation member 2400 may be movable with respect to each other (e.g., to adjust the length of the electrode portion 1410 of the first energy-delivering member 1400, such as in a manner described above with respect to the example of an embodiment illustrated in FIG. 4A and FIG. 4B), or static with respect to each other and well-bonded together (such as to increase stability, column strength, etc., and generally resulting in a fixed length of the electrode portion 1410). This portion of the energy-delivering assembly 410 may be substantially the same as or similar to the energy-delivering member 1000 and insulation member 2000 of the energy-delivering assembly 110 illustrated in FIG. 1 and FIG. 2. Accordingly, similar reference numbers, increased by 100, are used to indicate the same or similar elements and reference may be made to the above descriptions with respect to such elements in FIG. 1 for the sake of brevity and without intent to limit. Optionally, the outer diameter of the distal portion of the first energy-delivering member 1400 forming an electrode portion 1410 may have substantially the same outer diameter as the first insulation member 2400, similar to the example of an embodiment of an energy-delivering assembly 110′ illustrated in FIG. 3, reference being made thereto for further details for the sake of brevity and without intent to limit.

In the example of an embodiment of an energy-delivering assembly 410 illustrated in FIG. 6A, FIG. 6B, and FIG. 6C, the second energy-delivering member 3400 is formed from a generally tubular member through which the first energy-delivering member 1400 and the first insulation member 2400 extend. In accordance with various principles of the present disclosure, the second energy-delivering member 3400 may be electrically conductive to form a second electrode portion 3410. A second insulation member 4400 may be positioned over the second energy-delivering member 3400 to define a second electrode portion 3410 between the distal end 3400d of the second energy-delivering member 3400 (e.g., the distal end 3402d of the elongate body 3402 of the second energy-delivering member 3400) and the distal end 4400d of the second insulation member 4400 (e.g., the distal end 4402d of the elongate body 4402 of the second insulation member 4400), as illustrated in FIG. 6C. In accordance with various principles of the present disclosure, the second energy-delivering member 3400 may be formed of a hypotube, and the second insulation member 2400 may be formed of an insulative material similar to the second energy-delivering member 3300 and second insulation member 4300 of the energy-delivering assembly 310 described above with reference to FIG. 5. The second energy-delivering member 3400 may support the insulation member 4400 positioned therearound. However, in contrast, instead of the second energy-delivering member 3400 and the second insulation member 4400 being movable with respect to each other, the second energy-delivering member 3400 and the second insulation member 4400 may be generally static with respect to each other and well-bonded together. As such, the proximal electrode of the energy-delivering assembly 410 illustrated in FIG. 6A, FIG. 6B, and FIG. 6C, defined by the second energy-delivering member 3400 and the second insulation member 4400, is sufficiently stiff to protect the sharp distal tip 1404 of the first energy-delivering member 1400 and thereby to provide the FNA protective structure typically provided by a separate sheath. Moreover, a second energy-delivering member 3400 with a second insulation member 4400 therearound and bonded thereto may have better column strength than members which are movable with respect to each other, as the second insulation member 4400 would be mechanically supported by the second energy-delivering member 3400. However, it will be appreciated that it may be desirable for each of the elements 1400, 2400, 3400, 4400 of the energy-delivering assembly 410 to be movable with respect to one another for further adjustability of the energy-delivering assembly 410, such as in any of the above-described manners.

In use, the distal electrode, defined by the first energy-delivering member 1400 and the first insulation member 2400, are movable (e.g., slidable) with respect to the proximal electrode, formed by the second energy-delivering member 3400 and the second insulation member 4400. As may be appreciated, the lengths of the respective electrode portions 1410, 3410 of the distal and proximal electrodes may be fixed (by virtue of the first insulation member 2400 being fixed with respect to the first energy-delivering member 1000, and the second insulation member 2400 being fixed with respect to the second energy-delivering member 4400). However, the distal and proximal electrodes may be movable with respect to each other, such as by moving the first energy-delivering member 1400 distally with respect to the second energy-delivering member 3400, or moving the second energy-delivering member 3400 proximally with respect to the first energy-delivering member 1400. Relative movement of the distal and proximal electrodes adjusts the length of the insulation between the electrode portions 1410, 3410 to affect the overall length of the adjustable bipolar energy-delivering assembly 410, such as to affect the treatment volume of the energy-delivering assembly 410.

The example of an embodiment of an energy-delivering assembly 410 illustrated in FIG. 6A, FIG. 6B, and FIG. 6C may initially be delivered to a target site T with the sharp distal tip 1404 of the distal electrode defined by the first energy-delivering member 1400 positioned (e.g., proximally retracted) within the proximal electrode defined by the second energy-delivering member 3400, such as illustrated in FIG. 6A. In such configuration, the delivery device (e.g., endoscope) or patient anatomy through which the energy-delivering assembly 410 is delivered is protected from the sharp distal tip 1404. Once the energy-delivering assembly 410 (in particular, the distal end 3400d of the second energy-delivering member 3400) is positioned at the target site T (e.g., tissue), such as extended distally out the distal end of the delivery device, the distal end 3400d of the second energy-delivering member 3400 may be contacted with the target site T, such as illustrated in FIG. 6A. The sharp distal tip 1404 of the first energy-delivering member 1400 may then be extended distally out of the second energy-delivering member 3400 (e.g., distally beyond the distal end 3400d of the energy-delivering member 3400 and/or the distal end 3402d of the elongate body 3402 thereof) for puncture of the target site T, such as illustrated in FIG. 6B. At this stage, the second energy-delivering member 1400 need not be advanced into the target site T. However, as may be appreciated, the advancement of the first energy-delivering member 1400 into the target site T may facilitate advancement of the second energy-delivering member 3400 into the target site T thereafter by creating an initial puncture/opening in the target site T. The second energy-delivering member 3400 may then be advanced into the target site T, such as illustrated in FIG. 6C. With both electrode portions 1410, 3410 of the energy-delivering assembly 410 positioned as desired with respect to the target site T, the adjustable bipolar energy-delivering assembly 410 may be adjusted as desired (such as described above) for delivery and/or aspiration of materials through the lumen 1403 of the first energy-delivering member 1400 and/or delivery of energy to and between the first electrode portion 1410 and the second electrode portion 3410.

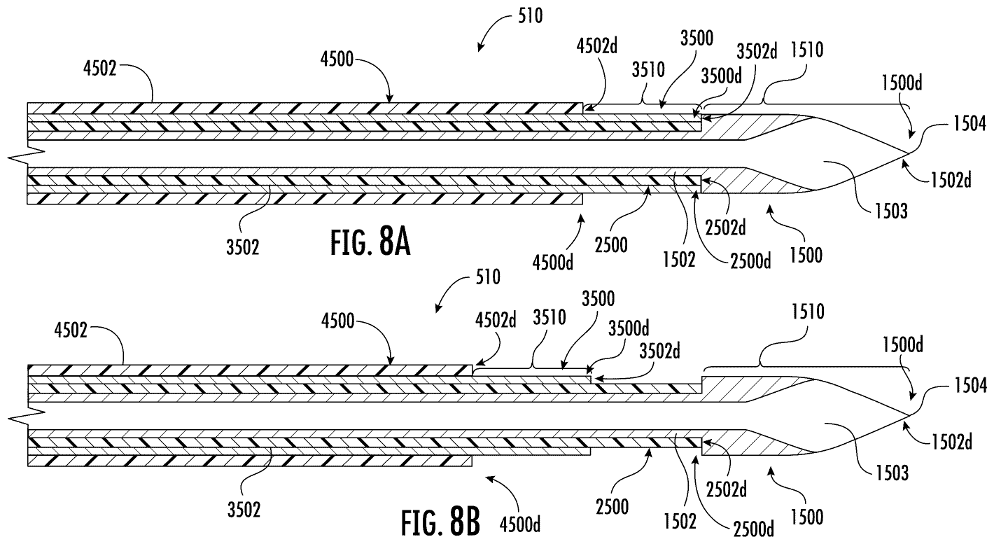

An example of an embodiment of a handle 440 which may be operatively coupled with one or more elements of the energy-delivering assembly 410 illustrated in FIG. 6A, FIG. 6B, and FIG. 6C, to control movement of the elements thereof, is illustrated in FIG. 7A, FIG. 7B, and FIG. 7C. Like the example of an embodiment of a handle 140 illustrated in FIG. 1, and the example of an embodiment of a handle 240 illustrated in FIG. 4A and FIG. 4B, the example of an embodiment of a handle 440 illustrated in FIG. 7A, FIG. 7B, and FIG. 7C has a first control section 442 operatively coupled with a sheath 420. The first control section 442 may be substantially similar (e.g., in form and/or function) to the first control section 142 described above with reference to FIG. 1, and/or the first control sections 242 described above with reference to FIG. 4A and FIG. 4B. Accordingly, similar reference numbers, increased by 300, are used to indicate the same or similar elements, and reference may be made to the above descriptions with respect to such elements in FIG. 1 for the sake of brevity and without intent to limit.

When the first energy-delivering member 1400 and the second energy-delivering member 2400 are positioned as in FIG. 6A, the various control sections 442, 444, 446, 448 of the handle 440 may be as illustrated in FIG. 7A. To set the distance between the first energy-delivering member 1400 and the second energy delivering member 3400 when in the positions illustrated in FIG. 6B and FIG. 6C (with the first energy-delivering member 1400 extended out of the second energy delivering member 3400), the user may adjust and set (e.g., fix in place) the position of an optional position control element 448 relative to the second control section 444. To advance the first energy-delivering member 1400 of the energy-delivering assembly 410 through the target site T (e.g., to puncture tissue/cells at the target site T), such as illustrated in FIG. 6B, the user advances (e.g., longitudinally/distally advances relative to the position illustrated in FIG. 7A) the second control section 444 (operatively coupled with the first energy-delivering member 1400) relative to the first control section 442 (operatively coupled with the sheath 420), such as by sliding the second control section 444 over a proximally-extending base or shaft 445, such as illustrated in FIG. 7B. In advancing the second control section 444 from the position illustrated in FIG. 7A to the position illustrated in FIG. 7B, the second control section 444 also moves distally with respect to the third control section 446 (operatively coupled with the second energy-delivering member 3400), thereby extending the first energy-delivering member 1400 distally from the second energy-delivering member 3400 so that the distal end 1400d of the first energy-delivering member 1400 is distal to the distal end 3400d of the second energy-delivering member 3400, such as illustrated in FIG. 6B and FIG. 7B. The sharp distal tip 1404 of the first energy-delivering member 1400 may be extended into the tissue along with the elongate body 1402 of the energy-delivering member 1400 and the insulation member 2400 surrounding the energy-delivering member 1400, such as illustrated in FIG. 6B. Optionally, the second control section 444 can be advanced distally (to advance the electrode 1410 into the patient) until it reaches a catch or the end of a slot in the handle 440, and/or the first control section 442. If a position control element 448 is present (and generally fixed with respect to the second control section 444), distal advancement of the second control section 444 advances the position control element 448 distally towards the third control section 446 until the position control element 448 abuts the third control section 446, as illustrated in FIG. 7B. Further advancing the second control section 444 relative to the position of FIG. 7B, as illustrated in FIG. 7C, also advances the position control element 448 which abuts and thus also advances the third control section 446 The full energy-delivering assembly 410 (including the second energy-delivering member 3400 and the insulation member 4400 therearound) is thereby advanced into the tissue, such as illustrated in FIG. 6C. An energy source may then be connected and delivered to the energy-delivering assembly 410, such as via a power connector 430 coupled to the proximal end 400p of the energy-delivering treatment system 400, such as in a manner described above with respect to the energy-delivering treatment system 100 illustrated in FIG. 1.

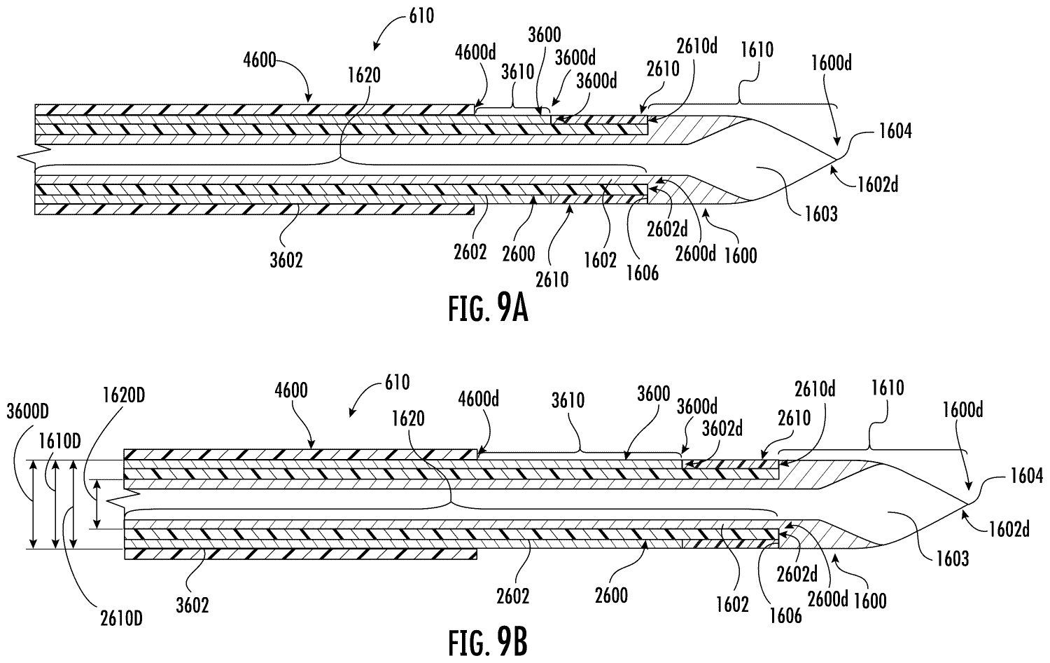

In accordance with various principles of the present disclosure, it may be desirable for an adjustable bipolar energy energy-delivering treatment system to have an energy-delivering assembly with electrodes having the same or substantially same outer dimensions (e.g., diameters), such as to facilitate a smoother puncture of tissue at the target site. In the example of an embodiment of an adjustable bipolar energy-delivering assembly 510 such as illustrated in FIG. 8A and FIG. 8B, a first (distal) energy-delivering member 1500 and a second (proximal) energy-delivering member 3500 have substantially the same outer dimensions (e.g., diameters). The initial puncture of tissue may be performed in a configuration of the energy-delivering assembly 510 such as illustrated in FIG. 8A, which may be considered a compressed configuration. Such configuration may avoid an overly flexible region between the electrode portion 1510 of the first energy-delivering member 1500 and the electrode portion 3510 of the second energy-delivering member 3500, thereby avoiding potential interference with puncturing which may be caused by an overly flexible device. It will be appreciated that various elements of the example of an embodiment of an energy-delivering assembly 510 illustrated in FIG. 8A and FIG. 8B are similar to elements of the example of an embodiment of an energy-delivering assembly 410 illustrated in FIGS. 6A-6C (other than, for instance, relative dimensions of the elements thereof). Accordingly, similar reference numbers, increased by 100, are used to indicate the same or similar elements and reference may be made to the above descriptions with respect to such elements in FIGS. 6A-6C for the sake of brevity and without intent to limit. Furthermore, it will be appreciated that the energy-delivering assembly 510 illustrated in FIG. 8A and FIG. 8B may be used in an energy-delivering treatment system 400 such as illustrated in FIGS. 7A-7C.

It will be appreciated that energy may be delivered to the energy-delivering assembly 510 in its configuration as illustrated in FIG. 8A. In particular, in a configuration as illustrated in FIG. 8A, the first (distal) electrode portion 1510 (formed by the first energy-delivering member 1500) and the second (proximal) electrode portion 3510 (formed by the second energy-delivering member 3500) may be in contact with each other and may be shorted together and activated in monopolar mode, as if they were a single electrode. In the illustrated example of an embodiment, with the outer diameter of the first energy-delivering member 1500 and the outer diameter of the second energy-delivering member 3500 being substantially equal, the first electrode portions 1510 and the second electrode portion 3510 may have the same or substantially the same diameters.

In some aspects, after the energy-delivering assembly 510 has been positioned as desired with respect to a target site, the first (e.g., distal) energy-delivering member 1500 and the second (e.g., proximal) energy-delivering member 3500 may be separated (e.g., moved with respect to each other). For instance, the second energy-delivering member 3500 may be moved proximally (e.g., retracted with respect to the first energy-delivering member 1500) and/or the first energy-delivering member 1500 may be advanced further distally with respect to the second energy-delivering member 3500, separating the first electrode portion 1510 and the second electrode portion 3510 by a distance determined by a medical professional to be appropriate for the treatment/procedure to be performed with the energy-delivering assembly 510 (e.g., delivery of energy to the energy-delivering assembly 510 to result in an IRE lesion which may cover a full tumor). It will be appreciated that the separation distance of the first electrode portion 1510 and the second electrode portion 3510 may be determined with considerations of avoiding electrical arcing therebetween as the electrode portions 1510, 3510 are activated in bipolar mode.

In some aspects, an outer sheath, such as any of the sheaths described above, may be provided over the energy-delivering assembly 510, and, particularly, over the sharp distal tip 1504 of the first energy-delivering member 1500 to protect the scope and/or anatomical passage through which the energy-delivering assembly 510 is advanced to the target site. In some aspects, the first insulation member 2500 positioned over the first energy-delivering member 1500 and/or the second insulation member 4500 positioned over the second energy-delivering member 3500 may be bonded with the respective underlying energy-delivering member, such as for increased column strength, etc., such as described above with respect to the example of an embodiment of an energy-delivering assembly 510 illustrated in FIGS. 6A-6C. However, it may be desirable for each of the elements 1500, 2500, 3500, 4500 to be movable with respect to one another for further adjustability of the energy-delivering assembly 510, such as in any of the above-described manners.

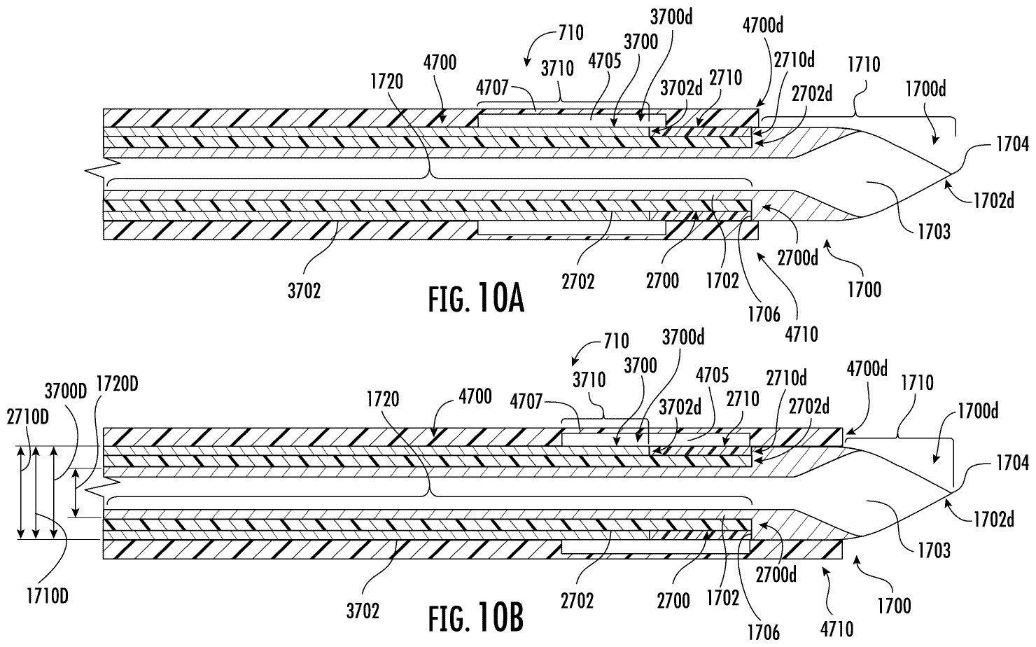

In some aspects, it may be desirable for at least one electrode portion of the energy-delivering assembly of a bipolar energy-delivering treatment system to be adjustable in addition to having the same or substantially the same outer dimensions (e.g., diameters) as the other of the electrode portions. In accordance with various principles of the present disclosure, an example of an embodiment of an adjustable bipolar energy-delivering assembly 610 with electrode portions having the same or substantially the same outer dimensions (e.g., diameters) is illustrated in FIG. 9A and FIG. 9B. Like the example of an embodiment of an energy-delivering assembly 510 illustrated in FIG. 8A and FIG. 8B, the first (distal) energy-delivering member 1600 and the second (proximal) energy-delivering member 3600 of the example of an embodiment of an energy-delivering assembly 610 illustrated in FIG. 9A and FIG. 9B have substantially the same outer dimensions (e.g., diameters). It will be appreciated that various elements of the energy-delivering assembly 610 illustrated in FIG. 9A and FIG. 9B are referenced with the same reference elements as in FIG. 8A and FIG. 8B, increased by 100 to indicate similar or same structures or features, and redundant description is generally omitted for the sake of brevity. However, unlike in the energy-delivering assembly 510 illustrated in FIG. 8A and FIG. 8B, the first electrode portion 1610 (defined by the first energy-delivering member 1600) and the second electrode portion 3610 (defined by the second energy-delivering member 3600) are maintained spaced apart by an insulation spacer member 2610. More particularly, the insulation spacer member 2610 maintains the first electrode portion 1610 spaced apart from the second electrode portion 3610 to maintain the energy-delivering assembly 610 as a bipolar energy-delivering assembly. Moreover, in some aspects, the outer diameter of the insulation spacer member 2610 is substantially the same as the first electrode portion 1610 and/or the second electrode portion 3610. In particular, the first energy-delivering member 1600 has an elongate body 1602 with a shoulder 1606 transitioning the elongate body 1602 from a first outer diameter 1610D of the electrode portion 1610 to a smaller second outer diameter 1620D of the stepped, reduced diameter proximal portion 1620 of the first energy-delivering member 1600 extending within (and insulated by) the first insulation member 2600. Such reduction in diameter of the first energy-delivering member 1600 allows for the second energy-delivering member 3600 to be extended over the first energy-delivering member 1600 as well as the first insulation member 2600 yet to have an outer diameter 3600D which is substantially the same as the outer diameter 3610D of the first electrode portion 1610. And, the outer diameter 2610D of the insulation spacer member 2610, positioned between the shoulder 1606 and the distal end 3602d of the elongate body 3602 of the second energy-delivering member 3600, is substantially the same as the outer diameter 3610D of the first electrode portion 1610. As such, similar to the example of an embodiment of an energy-delivering assembly 110′ illustrated in FIG. 3, the example of an embodiment of an energy-delivering assembly 610 illustrated in FIG. 9A and FIG. 9B may facilitate a smoother puncture of tissue by the sharp distal tip 1604 of the energy-delivering member elongate body 1602, followed by insertion of the first electrode portion 1610 and the second electrode portion 3610 into the tissue at the target site.

To allow for adjustability of the bipolar energy-delivering assembly 610 illustrated in FIG. 9A and FIG. 9B, a second insulation member 4600, positioned over the second energy-delivering member 3600, may be in the form of an insulated sheath slidable with respect to the second energy-delivering member 3600. The second insulation member 4600 may be distally advanced or proximally retracted with respect to the second energy-delivering member 3600 to change the exposed and effective length of the proximal electrode formed by the second energy-delivering member 3600, thereby adjusting the length of the ablation zone. In some aspects, the second insulation member 2460 is a thin-walled insulated sheath, such as made of polyimide tubing or other insulative materials, such as approximately 0.0005″ (0.0127 mm) thick up to approximately 0.005″ (0.127 mm) thick, depending upon material and dielectric requirements. In some aspects, an outer sheath, such as any of the sheaths described above, may be provided over the energy-delivering assembly 610, and, particularly, over the sharp distal tip 1604 of the first energy-delivering member 1600 to protect the scope and/or anatomical passage through which the energy-delivering assembly 610 is advanced to the target site.

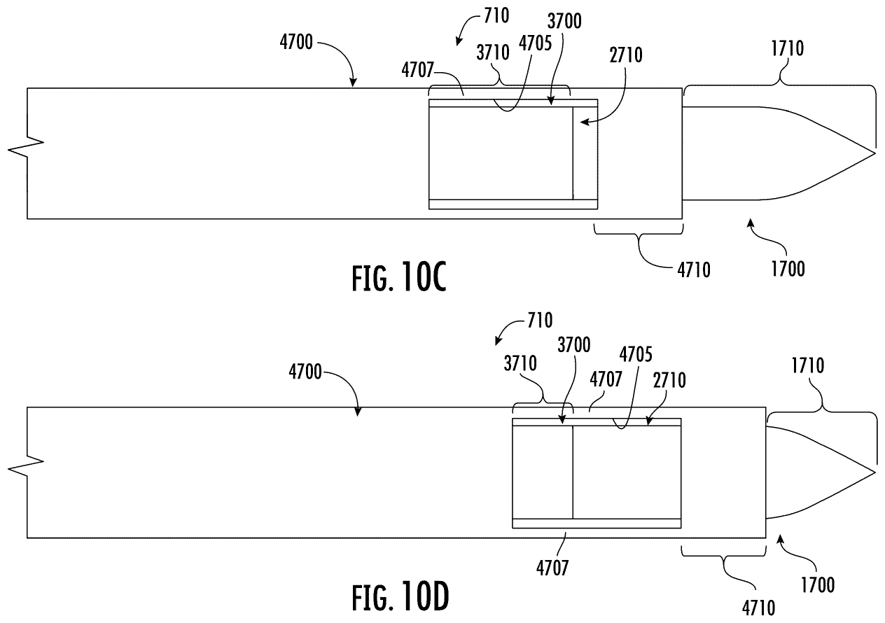

In some aspects, it may be desirable to adjust not only the proximal electrode portion of an adjustable bipolar energy-delivering assembly, but also the distal electrode portion of an adjustable bipolar energy-delivering assembly, with both electrode portions having substantially the same outer diameters. In the example of an embodiment of an energy-delivering assembly 710 illustrated in FIG. 10A, FIG. 10B, and FIG. 10C, both a first electrode portion 1710 formed by a first energy-delivering member 1700 and a second electrode portion 3710 formed by a second energy-delivering member 3700 are adjustable. Additionally, similar to the energy-delivering assembly 610 of FIG. 9A and FIG. 9B, the first electrode portion 1710 and the second electrode portion 3710 have substantially the same outer diameters. For the sake of convenience, various elements of the energy-delivering assembly 710 illustrated in FIG. 10A, FIG. 10B, FIG. 10C, and FIG. 10D are referenced with the same reference elements as in FIG. 9A and FIG. 9B, increased by 100 to indicate similar or same structures or features, and redundant description is generally omitted for the sake of brevity.

In the example of an embodiment of an energy-delivering assembly 710, as may be appreciated from the cross-sectional views of FIG. 10A and FIG. 10B, the first energy-delivering member 1700 is positioned within a first insulation member 2700 to be insulated from the second energy-delivering member 3700, the first electrode portion 1710 is separated and insulated from the second electrode portion 3710 by an insulation spacer member 2710, and a second insulation member 4700 is slidably positioned over both the first energy-delivering member 1700 and the second energy-delivering member 3700 to adjust both the first electrode portion 1710 and the second electrode portion 3710. As in the example of an embodiment illustrated in FIG. 9A and FIG. 9B, the insulation spacer member 2710 may have an outer diameter 2710D which is substantially equal to the first outer diameter 1710D of the first electrode portion 1710 of the first energy-delivering member 1700. Moreover, the first energy-delivering member 1700 may be stepped at a shoulder 1706 to a reduced second outer diameter 1720D along a proximal portion 1720 of the first energy-delivering member 1700. Such reduction in diameter allows for positioning of the second insulation member 2700 and the second energy-delivering member 3700 over the reduced-diameter proximal portion 1720 of the first energy-delivering member 1700 while allowing the outer diameter 3700D of the second energy-delivering member 3700 (and the second electrode portion 3710 defined by the second energy-delivering member 3700) to be substantially equal to the outer diameter 1710D of the first electrode portion 1710.

As may be more readily appreciated in the elevational view of the energy-delivering assembly 710 illustrated in FIG. 10C, a window 4705 is formed in the second insulation member 4700 to expose a portion of the second electrode portion 3710 therethrough. Legs 4707 alongside the window 4705 hold a distal insulation portion 4710 of the second insulation member 4700 over at least a portion of the first electrode portion 1710. In some instances, two legs 4707 are provided to form two windows about the circumferential extent of the second insulation member 4700. Alternatively, up to about 150° of the circumference of the second insulation member 4700 may be removed, leaving a single leg 4707 having an approximately 30° extent around the circumference of the second insulation member 4700 to hold the sections of the second insulation member 4700 on either axial side of the window 4705 in place. The second insulation member 4700 is positioned over both the first energy-delivering member 1700 and the second energy-delivering member 3700 to partially cover/insulate both members 1700, 3700, while exposing sections thereof to perform as electrodes 1710, 3710 (spaced apart by the insulation spacer member 2710) of the energy-delivering assembly 710. Distal advancement and proximal retraction of the second insulation member 4700 adjusts the exposed portions 1710, 3710 of the energy-delivering members 1700, 3700 simultaneously. The second insulation member 4700 may be made of polyimide tubing or other insulative materials, and may be a relatively thin-walled sheath (e.g., approximately 0.0005″ (0.0127 mm) thick up to approximately 0.010″ (0.254 mm) thick, depending upon material and dielectric requirement) in intimate contact with (e.g., in direct contact, yet still slidable with respect to) the energy-delivering members 1700, 3700. As with the example of an embodiment energy-delivering assembly 610 illustrated in FIG. 9A and FIG. 9B, an outer sheath, such as any of the sheaths described above, may be provided over the energy-delivering assembly 710, and, particularly, over the sharp distal tip 1704 of the first energy-delivering member 1700 to protect the scope and/or anatomical passage through which the energy-delivering assembly 710 is advanced to the target site.

It will be appreciated that each of the energy-delivering assemblies described above provides various structures, devices, systems, assemblies, and methods for providing energy-based treatment along with delivery of a treatment material through a lumen of an energy-delivering member and/or aspiration of materials from a target site through the lumen of the energy-delivering member. In some aspects, the above-described devices, systems, assemblies, and methods allow for adjustability of energy-delivering structures, devices, systems, and/or assemblies to vary the characteristics of the energy treatment being delivered. It will be appreciated that the devices, systems, assemblies, and methods disclosed herein may be delivered endoscopically, or percutaneously, as well as use within other steerable luminal access devices.

It will be appreciated that all structures, devices, systems, assemblies, and methods discussed herein are examples implemented in accordance with one or more principles of this disclosure, and are not the only way to implement these principles, and thus are not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An energy-delivering assembly comprising:
a first energy-delivering member having a sharp distal tip configured to puncture tissue, and a lumen defined therethrough and having a distal opening adjacent the sharp distal tip for delivery of materials distally through the lumen and out the distal opening thereof and/or for aspiration of materials into the distal opening of the lumen and proximally through the lumen; and
a first insulation member extending over said first energy-delivering member and having a distal end;
wherein a first electrode portion of said energy-delivering assembly is defined along said first energy-delivering member between the sharp distal tip thereof and the distal end of said first insulation member.

2. The energy-delivering assembly of claim 1, wherein said first electrode portion is adjustable by moving said first insulation member with respect to said first energy-delivering member.

3. The energy-delivering assembly of claim 1, wherein said first electrode portion and said first insulation member have substantially the same outer diameters.

4. The energy-delivering assembly of claim 1, further comprising:
a second energy-delivering member positioned over said first insulation member and having a distal end; and
a second insulation member extending over said second energy-delivering member and having a distal end;
wherein a second electrode portion of said energy-delivering assembly is defined along said second energy-delivering member between the distal end thereof and the distal end of said second insulation member.

5. The energy-delivering assembly of claim 4, wherein at least one of said first energy-delivering member, said first insulation member, said second energy-delivering member, or said second insulation member is movable with respect to another of said first energy-delivering member, said first insulation member, said second energy-delivering member, or said second insulation member to adjust at least one of the first electrode portion or the second electrode portion.

6. The energy-delivering assembly of claim 5, wherein said first insulation member is movable with respect to said first energy-delivering member to adjust the first electrode portion.

7. The energy-delivering assembly of claim 6, wherein said second energy-delivering member is movable with respect to said first insulation member.

8. The energy-delivering assembly of claim 7, wherein said second insulation member is movable with respect to said second energy-delivering member.

9. The energy-delivering assembly of claim 5, wherein said second energy-delivering member is movable with respect to said first insulation member.

10. The energy-delivering assembly of claim 5, wherein said second insulation member is movable with respect to said second energy-delivering member.

11. The energy-delivering assembly of claim 5, wherein said first energy-delivering member and said first insulation member are fixed with respect to each other, and said second energy-delivering member and said second insulation member are fixed with respect to each other.

12. The energy-delivering assembly of claim 11, wherein said second energy-delivering member is distally advanceable to move the distal end thereof distal to the distal end of said first energy-delivering member to sheathe the sharp distal tip of said first energy-delivering member.

13. The energy-delivering assembly of claim 4, wherein said first electrode portion and said second electrode portion have substantially the same outer diameters.

14. The energy-delivering assembly of claim 13, wherein said second insulation member is movable with respect to said second energy-delivering member.

15. The energy-delivering assembly of claim 14, wherein a window is defined in said second insulation member to expose varying lengths of said second electrode portion, and the distal end of said second insulation member is movable over said first electrode portion to expose varying lengths of said first electrode portion.

16. An energy-delivering treatment system comprising:
an energy-delivering assembly comprising:
a first energy-delivering member having a sharp distal tip configured to puncture tissue, and a lumen defined therethrough and having a distal opening adjacent the sharp distal tip for delivery of materials distally through the lumen and out the distal opening thereof and/or for aspiration of materials into the distal opening of the lumen and proximally through the lumen; and
a first insulation member extending over said first energy-delivering member and having a distal end;
wherein a first electrode portion of said energy-delivering assembly is defined along said first energy-delivering member between the sharp distal tip thereof and the distal end of said first insulation member; and
a handle, said energy-delivering assembly extending distally from said handle, wherein said handle:
has a first control section operatively coupled with said first insulation member to adjust the position of said first insulation member with respect to said first energy-delivering member; and
is configured to be operatively coupled with an energy source to supply energy to said first energy-delivering member.

17. The energy-delivering treatment system of claim 16, further comprising:
a sheath extending distally from said handle, said first energy-delivering member and said first insulation member extending distally from said handle through said sheath; and
a second control section operatively coupled with said first energy-delivering member to adjust a position thereof relative to said sheath.

18. The energy-delivering treatment system of claim 16, further comprising:
a second energy-delivering member positioned over said first insulation member and having a distal end;
a second insulation member extending over said second energy-delivering member and having a distal end; and
a third control section operatively coupled with said second insulation member to adjust a position thereof relative to said second energy-delivering member;
wherein a second electrode portion of said energy-delivering assembly is defined along said second energy-delivering member between the distal end thereof and the distal end of said second insulation member.

19. A method of treating a target site within a human patient, said method comprising:
delivering energy to a first energy-delivering member covered by a first insulation member to define a first electrode portion at a distal end of the first energy-delivering member; and
delivering materials through a lumen defined through the first energy-delivering member and with respect to the target site;
wherein delivering materials comprises at least one of delivering materials distally through the lumen to the target site or aspirating materials proximally through the lumen from the target site.

20. The method of claim 19, further comprising adjusting the length of the first electrode portion.
